(19) United States
(12) Patent Application Publication
(10) Pub. No.: US 2024/0058124 A1
(43) Pub. Date: Feb. 22, 2024

(54) DELIVERY DEVICE FOR PROSTHETIC HEART VALVE WITH AXIAL FORCE ADJUSTMENT DEVICE

(71) Applicant: Medtronic, Inc., Minneapolis (MN)

(72) Inventors: Stephen Montgomery, Galway (IE); Mark Ward, Roscommon (IE)

(21) Appl. No.: 18/270,111

(22) PCT Filed: Jan. 6, 2022
(86) PCT No.: PCT/US2022/011371

§ 371 Date: Jun. 28, 2023

Related U.S. Application Data
(60) Provisional application No. 63/134,252, filed on Jan. 6, 2021.

(51) Int. Cl. A61F 2/24; A61F 2/95; A61F 2/966

(52) U.S. Cl. CPC A61F 2/2436; A61F 2/9517; A61F 2/966; A61F 2002/9665

(57) ABSTRACT

Delivery devices and methods for delivering a prosthetic heart valve. The delivery device includes an inner shaft assembly with a valve retainer, an outer shaft assembly, a handle assembly and an axial force adjustment assembly. The outer shaft assembly includes a capsule containing the prosthetic valve in a loaded state. The handle assembly is coupled to a proximal region of the outer shaft assembly. The axial force adjustment assembly connects a proximal section of the inner shaft assembly to the handle assembly, and is configured to selectively move the proximal section relative to the handle assembly. The axial force adjustment assembly can include an actuator member and a driver member. The driver member directly interfaces with the inner shaft assembly, and a force applied to the actuator member is transferred onto the proximal section via the driver member. A sensor for sensing tension and/or compression in the inner shaft assembly can be provided.

### FIELD

The present disclosure relates to transcatheter prosthesis delivery devices and methods. More particularly, it relates to devices and methods for percutaneously delivering a stented prosthetic heart valve with adjustment for axial forces (tension/compression).

### BACKGROUND

Diseased or otherwise deficient heart valves can be repaired or replaced with an implanted prosthetic heart valve. Conventionally, heart valve replacement surgery is an open-heart procedure conducted under general anesthesia, during which the heart is stopped and blood flow is controlled by a heart-lung bypass machine. Traditional open surgery inflicts significant patient trauma and discomfort, and exposes the patient to a number of potential risks, such as infection, stroke, renal failure, and adverse effects associated with the use of the heart-lung bypass machine, for example.

Due to the drawbacks of open-heart surgical procedures, there has been an increased interest in minimally invasive and percutaneous replacement of cardiac valves. With percutaneous transcatheter (or transluminal) techniques, a prosthetic heart valve is compacted for delivery in a catheter and then advanced, for example, through an opening in the femoral artery and through the descending aorta to the heart, where the prosthetic heart valve is then deployed in the annulus of the valve to be restored (e.g., the aortic valve annulus). Although transcatheter techniques have attained widespread acceptance with respect to the delivery of conventional stents to restore vessel patency, only mixed results have been realized with percutaneous delivery of the more complex prosthetic heart valve.

The heart valve prosthesis employed with transcatheter procedures generally includes an expandable multi-level frame or stent that supports a valve structure having a plurality of leaflets. The frame can be contracted during percutaneous transluminal delivery, and expanded upon deployment at or within the native valve. With some stented prosthetic heart valve designs, the stent frame is formed to be self-expanding. The stented valve is compressed or crimped down to a desired size and held in that compressed state within a sheath for transluminal delivery. Retracting the sheath from this valved stent allows the stent to self-expand to a larger diameter, fixating at the native valve site. During this deployment action, axial forces (tension or compression) can develop as segments of the stented prosthetic heart valve are released from the confines of the sheath, begin to expand, and engage native tissue. Forces will act on the partially deployed stented prosthetic heart valve/native tissue interface due, for example, to pulling or pushing of the delivery device. This tension/compression can impact the accuracy of valve deployment. If the stented prosthetic heart valve is under tension during deployment, there can be a risk of valve “popup” during release. If the stented prosthetic heart valve is under compression, there can also be a risk of valve movement during release. It can be difficult to understand what compression is on the stented prosthetic heart valve as compression forces are being absorbed throughout the anatomy proximal a location of the stented valve itself. Likewise, it can be difficult to understand tension on the stented prosthetic heart valve.

### SUMMARY

The inventors of the present disclosure have recognized a need to address one or more of the above-mentioned problems. The details of one or more aspects of the disclosure are set forth in the accompanying drawings and the description below. Other features, objects, and advantages of the techniques described in this disclosure will be apparent from the description and drawings, and from the claims.

Some aspects of the present disclosure relate to a delivery device for percutaneously delivering a stented prosthetic heart valve. The delivery device includes an inner shaft assembly, an outer shaft assembly, a handle assembly and an axial force adjustment assembly. The inner shaft assembly includes a valve retainer configured for temporary connection to a stented prosthetic heart valve in a loaded state of the delivery device. The outer shaft assembly is co-axially received over the inner shaft assembly and includes a capsule configured to contain the stented prosthetic heart valve in the loaded state. The handle assembly is coupled to a proximal region of the outer shaft assembly. The axial force adjustment assembly connects a proximal section of the inner shaft assembly to the handle assembly. Further, the axial force adjustment assembly is configured to selectively move the proximal section of the inner shaft assembly relative to the handle assembly. In some embodiments, the axial force adjustment assembly is configured to selectively alter an axial force in the inner shaft assembly, the axial force being one of tension and compression. In some embodiments, the axial force adjustment assembly includes an actuator member linked to a driver member. The driver member directly interfaces with the proximal section of the inner shaft assembly, and the axial force adjustment assembly is configured to selectively transfer a force applied to the actuator member onto the proximal section via the driver member.

Other aspects of the present disclosure related to a method for restoring a defective heart valve in a patient. The method can include manipulating a delivery device loaded with a radially expandable stented prosthetic heart valve in a radially compressed condition. In this regard, the delivery device includes an outer shaft assembly including a capsule containing the stented prosthetic heart valve, an inner shaft assembly co-axially disposed within the outer shaft assembly and including a valve retainer connected to a proximal segment of the stented prosthetic heart valve, a handle assembly coupled to a proximal region of the outer shaft assembly, and an axial force adjustment assembly connecting a proximal section of the inner shaft assembly to the handle assembly. The step of manipulating includes guiding the stented prosthetic heart valve through a vasculature of the patient and into the defective heart valve by moving the handle assembly, which correspondingly moves the outer shaft assembly and the inner shaft assembly. The capsule is partially retracted to expose a distal segment of the stented prosthetic heart valve such that the exposed distal segment radially expands. The steps of manipulating and retracting generate an axial force in the inner shaft assembly. The axial force adjustment assembly is operated to lessen the axial force in the inner shaft assembly. The stented prosthetic heart valve is released from the delivery device. In some embodiments, the step of operating include rotating an actuator member of the axial force adjustment assembly. In some embodiments, following the step of partially retracting, the exposed distal segment engages native anatomy at the defective heart valve and generates stress in the stented prosthetic heart valve, and a component force of the stress is transferred to the inner shaft assembly via the valve retainer to generate the axial force in the inner shaft assembly. In some embodiments, prior to the step of operating, the method further includes evaluating an axial force in the inner shaft assembly based upon information from a sensor located along the inner shaft assembly.

Other aspects of the present disclosure relate to a delivery device for percutaneously delivering a stented prosthetic heart valve. The delivery device includes an inner shaft assembly, an outer shaft assembly, a handle assembly, and a sensor. The inner shaft assembly includes a valve retainer configured for temporary connection to a stented prosthetic heart valve in a loaded state of the delivery device. The outer shaft assembly is co-axially received over the inner shaft assembly and includes a capsule configured to contain the stented prosthetic heart valve in the loaded state. The handle assembly is coupled to a proximal region of the outer shaft assembly. The sensor configured and arranged to sense a parameter indicative of an axial force in the inner shaft assembly. In some embodiments, information generated by the sensor is displayed to a user.

### BRIEF DESCRIPTION OF DRAWINGS

FIG. 1 is a perspective view of a delivery device in accordance with principles of the present disclosure and loaded with a stented prosthetic heart valve;

FIG. 2 is a simplified, cross-sectional view, with portions shown in block form, of the loaded delivery device of FIG. 1;

FIG. 3A is an enlarged perspective view of a portion of the delivery device of FIG. 1;

FIG. 3B is a cross-sectional view of the portion of the delivery device of FIG. 3A;

FIG. 4 illustrates use of the delivery device of FIG. 1 in delivering a prosthetic heart valve to, and deploying the prosthetic heart valve at, at native heart valve target site.

FIG. 5A is a simplified cross-sectional view of a delivery device in accordance with principles of the present disclosure, including an axial force adjustment assembly in an engaged state;

FIG. 5B is an enlarged view of a portion of the delivery device of FIG. 5A;

FIG. 5C is the simplified cross-sectional view of the delivery device of FIG. 5A and illustrating the axial force adjustment assembly in a disengaged state;

FIG. 6A is a simplified cross-sectional view of a delivery device in accordance with principles of the present disclosure;

FIG. 6B is an enlarged view of a portion of the delivery device of FIG. 6A;

FIG. 7A is a side view of a stented prosthetic heart valve useful with the systems, devices and methods of the present disclosure and in a normal, expanded condition; and

FIG. 7B is a side view of the prosthetic heart valve of FIG. 7A in a compressed condition.

### DETAILED DESCRIPTION

Specific embodiments of the present disclosure are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

As referred to herein, stented transcatheter prosthetic heart valves useful with and/or as part of the various systems, devices and methods of the present disclosure may assume a wide variety of different configurations, such as a bioprosthetic heart valve having tissue leaflets or a synthetic heart valve having polymeric, metallic or tissue-engineered leaflets, and can be specifically configured for replacing any of the four valves of the human heart. Thus, the stented prosthetic heart valve useful with the systems, devices, and methods of the present disclosure can be generally used for replacement of a native aortic, mitral, pulmonic or tricuspid valve, or to replace a failed bioprosthesis, such as in the area of an aortic valve or mitral valve, for example. In yet other embodiments, the devices, systems, and methods of the present disclosure can be useful for delivering other stented prostheses that may or may not be a stented prosthetic heart valve.

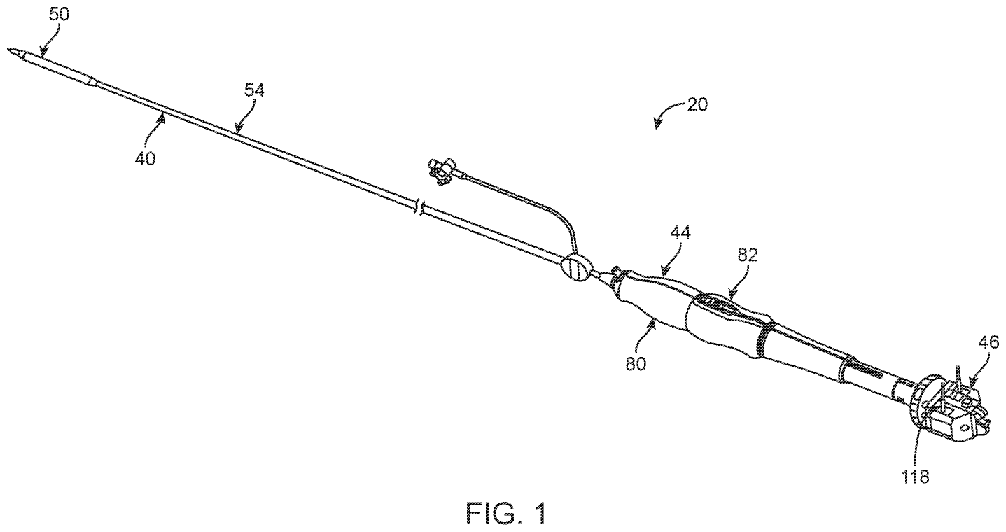

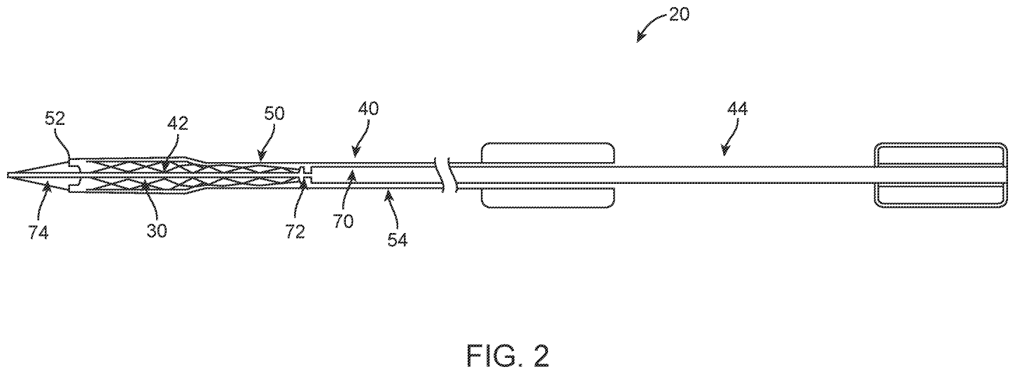

One example of a delivery device 20 in accordance with principles of the present disclosure is provided in FIG. 1. Portions of the delivery device 20 are further illustrated in simplified form in FIG. 2, and loaded with a stented prosthesis 30, such as a stented prosthetic heart valve. The delivery device (or delivery catheter system) 20 includes an outer shaft assembly 40, an inner shaft assembly 42, a handle assembly 44, and an axial force adjustment assembly 46 (referenced generally in FIG. 1, hidden in FIG. 2). Details on the various components are provided below. In general terms, however, the delivery device 20 combines with the stented prosthetic heart valve 30 to form a system for performing a therapeutic procedure on a defective heart valve of a patient. The delivery device 20 provides a loaded or delivery state (shown in FIGS. 1 and 2) in which the stented prosthetic heart valve 30 is loaded over the inner shaft assembly 42 and is compressively retained within a capsule 50 of the outer shaft assembly 40. The outer shaft assembly 40 can be manipulated to withdraw the capsule 50 proximally from over the prosthetic heart valve 30 via operation of the handle assembly 44, permitting the prosthesis 30 to self-expand and release from the inner shaft assembly 42. The delivery device 20 can optionally include other components. For example, the delivery device 20 can include one or more features for sensing axial forces (tension or compression) along the inner shaft assembly 42, and displaying information indicative of the sensed axial forces to a user as described below.

The axial force adjustment assembly 46 is operable to selectively alter an axial force (tension or compression) along the inner shaft assembly 42, for example by selectively moving a proximal section of the inner shaft assembly 42 relative to the handle assembly 44. To the extent the inner shaft assembly 42 is under tension, distal movement will lessen the applied tension; to the extent the inner shaft assembly 42 is being compressed, proximal movement will lessen the applied compressive force. Regardless, under circumstances where the stented prosthetic heart valve 30 is under tension/compression while connected to the inner shaft assembly 42 (e.g., during a deployment procedure, the capsule 50 may be partially retracted from a distal segment of the stented prosthetic heart valve 30 while a proximal segment of the prosthetic heart valve 30 remains connected to the inner shaft assembly 42), this tension/compression can be lessened by adjusting tension/compression in the inner shaft assembly 42.

Various features of the components 40-44 reflected in FIGS. 1 and 2 can be modified or replaced with differing structures and/or mechanisms. Thus, the present disclosure is in no way limited to the outer shaft assembly 40, the inner shaft assembly 42, or the handle assembly 44 as shown and described below. Any construction that generally facilitates compressed loading of a stented prosthetic heart valve over an inner shaft via a retractable outer sheath or capsule is acceptable. Further, the delivery device 20 can optionally include additional components or features not shown.

In some embodiments, the outer shaft assembly 40 extends from the handle assembly 44 to a distal end 52, and includes the capsule 50 and an outer shaft 54. The outer shaft assembly 40 can be akin to a catheter, defining a lumen that extends from the distal end 52 through the capsule 50 and at least a portion of the outer shaft 54. The capsule 50 extends distally from the outer shaft 54, and in some embodiments has a more stiffened construction (as compared to a stiffness of the outer shaft 54) that exhibits sufficient radial or circumferential rigidity to overtly resist the expected expansive forces of the stented prosthetic heart valve 30 when compressed within the capsule 50. For example, the outer shaft 54 can be a polymer tube embedded with a metal braiding, whereas the capsule 50 includes a laser-cut metal tube that is optionally embedded within a polymer covering. Alternatively, the capsule 50 and the outer shaft 54 can have a more uniform or even homogenous construction (e.g., a continuous polymer tube). Regardless, the capsule 50 is constructed to compressively retain the stented prosthetic heart valve 30 at a predetermined diameter when loaded within the capsule 50, and the outer shaft 54 serves to connect the capsule 50 with the handle assembly 44. The outer shaft 54 (as well as the capsule 50) is constructed to be sufficiently flexible for passage through a patient's vasculature, yet exhibits sufficient longitudinal rigidity to effectuate desired axial movement of the capsule 50. In other words, proximal retraction of the outer shaft 54 is directly transferred to the capsule 50 and causes a corresponding proximal retraction of the capsule 50. In other embodiments, the outer shaft 54 is further configured to transmit a rotational force or movement onto the capsule 50.

The inner shaft assembly 42 can have various constructions appropriate for supporting the outer shaft assembly 40, including supporting the prosthetic heart valve 30 disposed thereon relative to the capsule 50. In some embodiments, the inner shaft assembly 42 includes an inner shaft 70 (i.e., a singular, continuous tubular shaft; two or more differently constructed tubular shafts that are connected to one another; etc.). Regardless, the inner shaft assembly 44 may form or define at least one lumen (not shown) sized, for example, to slidably receive a guide wire (not shown).

The inner shaft assembly 42 further includes, or is connected to or includes a valve retainer or mechanism 72 and a tip 74. The valve retainer 72 can assume various forms and is configured to selectively capture or retain corresponding feature of the prosthetic heart valve 30 (thus retaining the prosthetic heart valve 30 relative to the inner shaft assembly 42 in the loaded state). In some non-limiting embodiments, for example, the valve retainer 72 includes one or more fingers sized to be received within corresponding apertures formed by a stent or frame of the prosthetic heart valve 30. Alternatively or in addition, the valve retainer 72 can be configured to selectively receive a corresponding feature (e.g., posts) provided with the prosthetic heart valve 30. The valve retainer 72 can have a spindle-like construction in some embodiments. Regardless, the valve retainer 72 is attached to or carried by the shaft(s) of the inner shaft assembly 42. When the capsule 50 is retracted proximally beyond the valve retainer 72, the stented prosthetic heart valve 30 can completely release or deploy from the delivery device 20. The delivery device 70 can optionally include other components that assist or facilitate or control complete deployment. The tip 74 forms or defines a nose cone having a distally tapering outer surface adapted to promote atraumatic contact with bodily tissue. The tip 74 can be fixed or slidable relative to the inner shaft 70.

The handle assembly 44 generally includes a housing or chassis 80 and one or more deployment actuator mechanisms (i.e., controls) 82 (referenced generally). The housing 80 can have any shape or size appropriate for convenient handling by a user. The housing 80 maintains the actuator mechanism(s) 82, with the handle assembly 44 configured to facilitate sliding movement of the outer shaft assembly 40 relative to the inner shaft assembly 42 via operation of the deployment actuator mechanism(s) 82. For example, the deployment actuator mechanism(s) 82 can be manipulated or moved (e.g., rotated) relative to the housing 80.

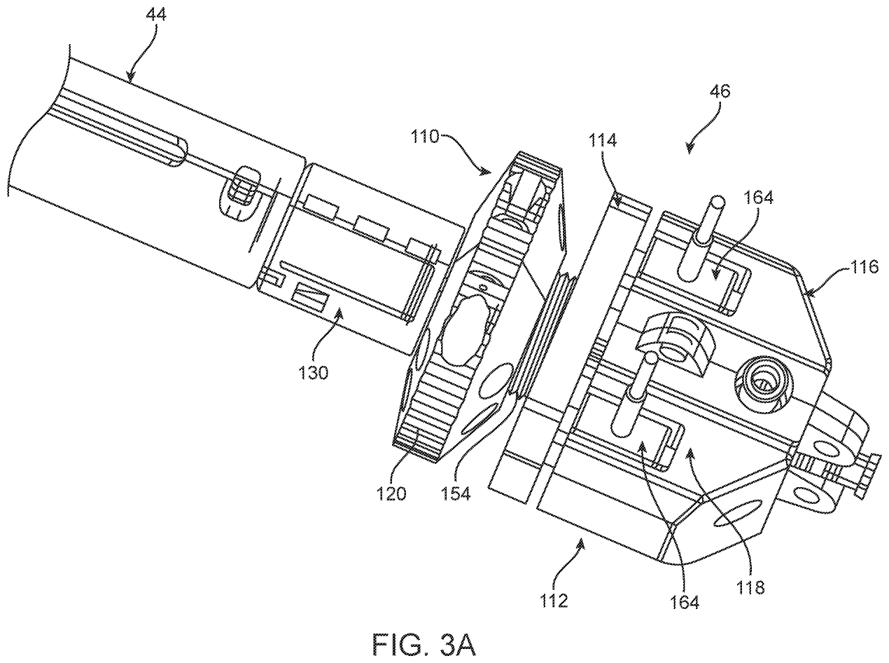

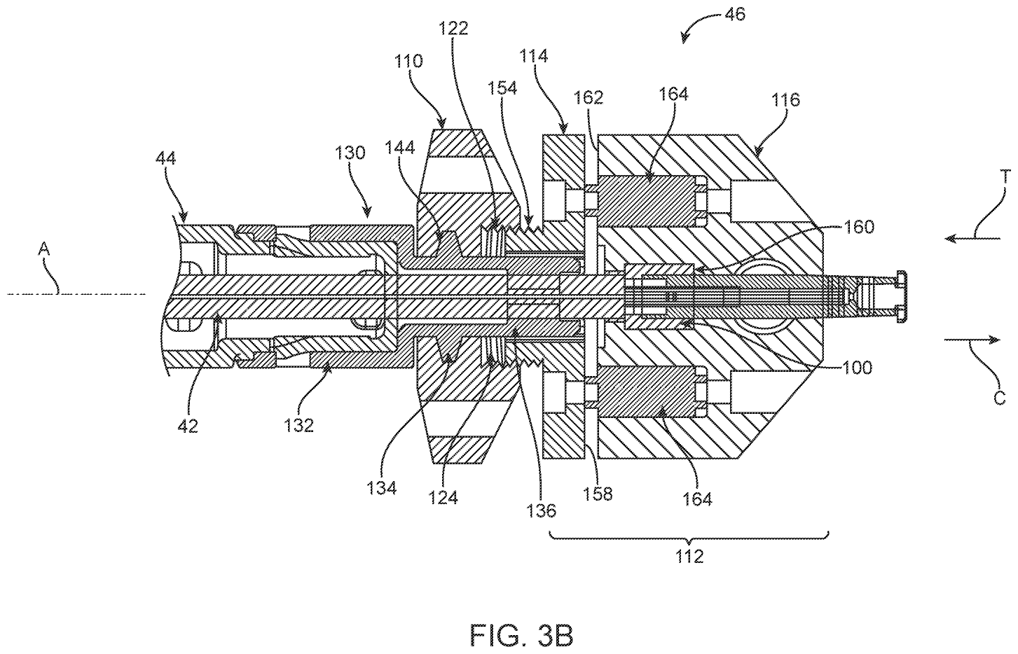

With the above general explanations of exemplary embodiments of the components 40-44 in mind, portions of one embodiment of the axial force adjustment assembly 46 is shown in greater detail in FIGS. 3A and 3B. As a point of reference, portions of the handle assembly 44 are visible in FIGS. 3A and 3B, as is a portion of the inner shaft assembly 42 in FIG. 3B. The tension adjustment assembly 46 connects a proximal section 100 of the inner shaft assembly 42 to the handle assembly 44, and includes an actuator member 110 and a driver member 112. In some embodiments, the driver member 112 can include two or more components, such as an attachment body 114 and a load transfer body 116. Regardless, the actuator member 110 is linked to the driver member 112, and the driver member 112 (e.g., the load transfer body 116) directly interfaces with the proximal section 100. With this in mind, the axial force adjustment assembly 46 is configured to selectively transfer a force applied to the actuator member 110 onto the proximal section 100 via the driver member 112, effecting a change in axial tension/compression. FIGS. 3A and 3B further illustrate an optional sensor unit 118 (referenced generally in FIG. 3A) described in greater detail below for sensing tension/compression in the inner shaft assembly 42. As a point of reference, the terms “axial” and “longitudinal” are in reference to a center, longitudinal axis A defined by the inner shaft assembly 42 as identified in FIG. 3B.

The actuator member 110 can assume various forms conducive to receiving a user-applied force upon final assembly, and in some embodiments is akin to a disk or wheel. In some embodiments, the actuator member 110 is sized and shaped to define a footprint or outer dimension(s) greater than that of the handle assembly 44 (at least in a region adjacent the actuator member 110) such that a contact surface 120 of the actuator member 110 is located beyond the handle assembly 44 and thus easily accessed by a user's hand/finger. For example, an outer diameter or dimension (i.e., dimension perpendicular to the longitudinal axis A) of the actuator member 110 at the contact surface 120 is greater than an outer diameter or dimension of the handle assembly 44 (at least in a region adjacent the actuator member 110). The contact surface 120 can optionally formed a knurled surface and/or incorporate other features that facilitate user interaction with the actuator member 110. Regardless, the actuator member 110 forms or incorporates one or more features that promote connection to the driver member 112. For example, the actuator member 110 can form or define an internally threaded surface 122 about a cavity 124 that is otherwise open to a trailing side of the actuator member 110. As described below, the attachment body 110 can include or form a complementary externally threaded surface for threadably engaging the internally threaded surface 122. Other connection formats are also acceptable.

The actuator member 110 can be retained relative to the handle assembly 44 in various manners. In one non-limiting example, the axial force adjustment assembly 46 further includes an end cap 130. The end cap 130 is a generally tubular body forming a lumen sized to receive the inner shaft assembly 40. Further, the end cap 130 defines a leading section 132, an intermediate section 134 and a trailing section 136. The leading section 132 is configured for attachment to the handle assembly 44, and can form or define a cup or similarly-shape structure size to be fitted over/attached to a proximal end 140 of the handle assembly 44. The intermediate section 134 provides one or more features for connection to the actuator member 110. For example, the intermediate section 134 can form or define a radially extending flange sized and shaped to be received within a corresponding groove 144 defined by the actuator member 110. With this construction, the actuator member 110 can rotate about the end cap 130, but is longitudinally captured relative to the end cap 130 at the flange/groove 144 interface. Other connection formats are also acceptable. The trailing section 136 forms one or more features that serve to centrally capture the inner shaft assembly 42, with the end cap 130, in turn, centrally maintaining the inner shaft assembly 42 relative to the handle assembly 44. For example, an inner diameter of the end cap 130 can be reduced along the trailing section 136, selected to approximate an outer diameter of a corresponding region of the inner shaft assembly 42. With these and related configurations, the end cap 130 is generally centered about the inner shaft assembly 42 in a manner permitting longitudinal movement or sliding of the inner shaft assembly 42 relative to the end cap 130.

The driver member 112 can assume various forms, and in some embodiments can include the attachment body 114 and the load transfer body 116 as mentioned above. The attachment body 114 is generally configured for connection with the actuator member 110, and can have a ring-like shape defining a central aperture sized to slidably receive the trailing section 136 of the end cap 130. A leading region of the attachment body 114 forms or defines an externally threaded surface 154 sized and shaped to threadably interface with the internally threaded surface 122 of the actuator member 110. A trailing region of the attachment body 114 has an increased outer diameter as compared to an outer diameter of the leading region, and forms or terminates at a trailing face 158 for reasons made clear below.

The load transfer body 116 is generally configured to support or retain the proximal section 100 of the inner shaft assembly 42, as well as other optional components. For example, in some embodiments, the load transfer body 116 forms a cavity 160 (referenced generally in FIG. 3B) open to a leading face 162. The cavity 160 is sized and shaped to longitudinally capture the proximal section 100. The load transfer body 116 can be connected to the attachment body 114 in various manners, for example by one or more pins (not shown). In some embodiments, the load transfer body 116 carries one or more load cells 164 that otherwise serve as, or as part of, the optional sensor unit 118. A head of each of the load cells 164 projects beyond the leading face 162 and, upon final assembly, is in contact with the trailing face 158 of the attachment body 114. With this arrangement, the load cell(s) 164 sense information indicative of axial tension/compression across the driver member 112, and thus at the inner shaft assembly 42 as described below. Other arrangements of the load transfer body 116 relative to the attachment body 114 are also acceptable.

Upon final assembly, the inner shaft assembly 42 extends through the end cap 130, and the proximal portion 100 is secured to the load transfer body 116 such that the proximal portion 100 moves axially or longitudinally with axial or longitudinal movement of the load transfer body 116. The load transfer body 116 is connected to the attachment body 114 in an axially or longitudinally fixed manner. The actuator member 110 is axially or longitudinally fixed relative to the handle assembly 44 via the end cap 130 (but can rotate relative to the end cap 130 and thus relative to the handle assembly 44). An axial or longitudinal link between the actuator member 110 and the attachment body 114 is established via threaded engagement at the threaded surfaces 122, 154. With this connection, the actuator member 110 dictates an axial or longitudinal position of the attachment body 114 relative to the handle assembly 44. Rotation of the actuator member 110 about the end cap 130 causes the attachment body 114 to translate axially or longitudinally. The load transfer body 116, and thus the captured proximal portion 100 of the inner shaft assembly 42, translates axially or longitudinally with the attachment body 114.

During use, the load transfer body 116 axially or longitudinally retains the proximal portion 100 of the inner shaft assembly 42 relative to the handle assembly 44 (and thus relative to the outer shaft assembly 40 (FIG. 2) that is otherwise secured to the handle assembly 44) via axial or longitudinal connections between the load transfer body 116/attachment body 114, the attachment body 114/actuator member 110, the actuator member 110/end cap 130, and the end cap 130/handle assembly 44. Axial forces (tension or compression) being experienced by the inner shaft assembly 42 are thus transferred to the driver member 112. Where provided, the load cells 164 measure or sense the so-translated tension/compression. Regardless, the axial force adjustment assembly 46 can be operated to lessen the tension or compression in the inner shaft assembly 42 by axially or longitudinally moving the proximal portion 100 relative to the handle assembly 46. For example, and with specific reference to FIG. 3B, tension in the inner shaft assembly 42 imparts a tension force T at the proximal portion 100/driver member 112 interface, and can be lessened by rotating the actuator member 110 in a direction that causes the driver member 112 to move the proximal portion 100 axially in a distal direction or toward the handle assembly 44 (leftward relative to the orientation of FIG. 3B). Conversely, compression in the inner shaft assembly 42 imparts a compression force C at the proximal portion 100/driver member 112 interface, and can be lessened by rotating the actuator member 110 in a direction that causes the driver member 112 to move the proximal portion 100 axially in a proximal direction or away from the handle assembly 44 (rightward relative to the orientation of FIG. 3B). Because axial forces (tension/compression) in the inner shaft assembly 42 can be directly related to or generated as a function of tension/compression at the prosthetic heart valve 30 (FIG. 2) during a delivery procedure, adjustment or lessening of axial forces on the inner shaft assembly 42 via operation of the axial force adjustment assembly 46 equates to a reduction of tension/compression in the prosthetic heart valve 30.

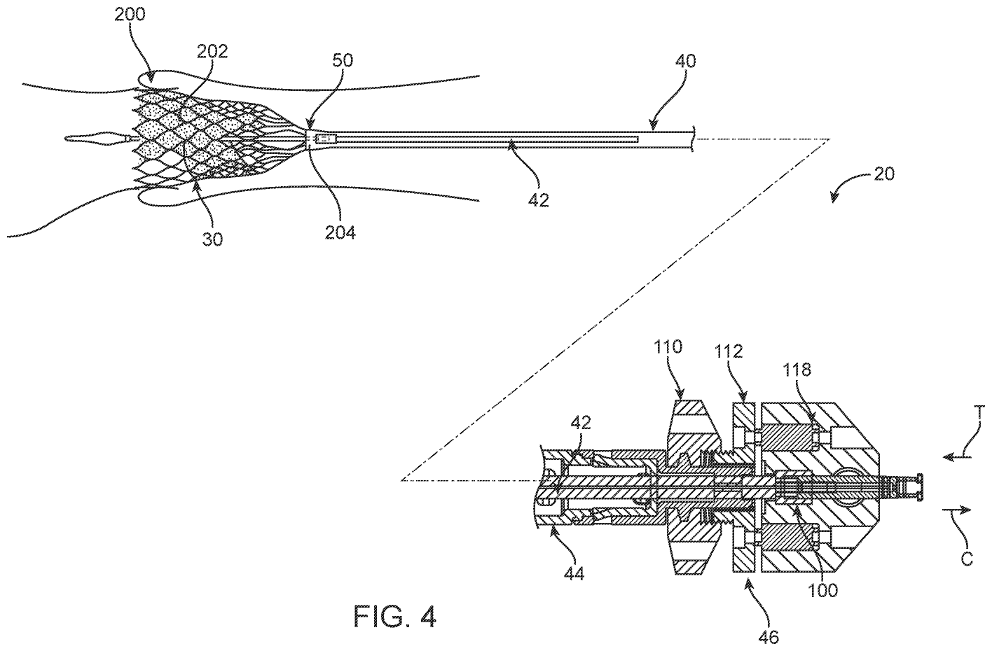

FIG. 4 illustrates portions of the delivery device 20 as part of a method of restoring a targeted defective heart valve 200 (referenced generally) in accordance with principles of the present disclosure, including delivering and deploying the prosthetic heart valve 30 at the target site 200 (e.g., at an annulus of the native heart valve 200). At the stage in the procedure of FIG. 4, the delivery device 20 has been loaded with the prosthetic heart valve 30 as described above, and the delivery device 20 has been manipulated to guide the prosthetic heart valve 30 through vasculature of the patient and into the target site 200. Further, the outer shaft assembly 40, and in particular the capsule 50, has been partially retracted from over the prosthetic heart valve 30 so as to expose a distal segment 202 of the prosthetic heart valve 30. With this arrangement, the exposed distal segment 202 has radially expanded. A proximal segment 204 (referenced generally) of the prosthetic heart valve 30 remains within the capsule 50, and is connected to the inner shaft assembly 42 (e.g., at the valve retainer 72 (FIG. 2)). In some instances, the clinician may manipulate the handle assembly 44 during retraction of the capsule 50 in an effort to move the partially-deployed prosthetic heart valve relative to the target site 200.

During the delivery and deployment procedures as described above, axial forces (tension or compression) can develop as segments of the stented prosthetic heart valve 30 are released from the confines of the capsule 50, begin to expand, and engage native tissue at the target site 200. Axial forces will act upon, or be generated within, the partially deployed stented prosthetic heart valve/native tissue interface due, for example, to pulling or pushing of the delivery device 20. For example, engagement between the expanding, exposed distal segment 202 and native anatomy may tend to “pull” the prosthetic heart valve 30 distally (leftward relative to the orientation of FIG. 4); because the proximal segment 204 remains stationary (i.e., the proximal segment 204 remains secured to the inner shaft assembly 42 at the valve retainer 72 (FIG. 2) and the inner shaft assembly 42 is otherwise being held stationary (due to connection to the handle assembly 44 that is being held by the clinician)), this “pulling” force generates tension in the prosthetic heart valve 30. Tension can also be created, for example, where the exposed distal segment 202 is engaged with native anatomy and the clinician applies a proximal pulling force onto the handle assembly 44; under these circumstances, the clinician-applied pulling/proximal force is imparted onto the proximal segment 204 at the valve retainer 72 while the distal segment 202 is prevented from moving proximally by the native anatomy. Regardless, tension in the prosthetic heart valve 30 is similarly experienced by the inner shaft assembly 42. Conversely, a clinician-applied, forward or distal “push” on the handle assembly 44 may create compression in the prosthetic heart valve 30. For example, the distal pushing force is transferred to the proximal segment 204 at the valve retainer 72 interface, while the exposed distal segment 202 is prevented from moving distally by the native anatomy.

Regardless of how created, the axial force adjustment assembly 46 can be operated by the clinician to lessen axial forces being experienced by the inner shaft assembly 42, and thus by prosthetic heart valve 30. For example, tension T in the inner shaft assembly 42 can be lessened by rotating the actuator member 110 in a direction that causes the driver member 112 to move the proximal portion 100 axially in a distal direction or toward the handle assembly 44 (leftward relative to the orientation of FIG. 4). Distal movement of the proximal portion 100 reduces tension in the inner shaft assembly 42; as a result, tension in the prosthetic heart valve 30 is also lessened. Conversely, compression C in the inner shaft assembly 42 can be lessened by rotating the actuator member 110 in a direction that causes the driver member 112 to move the proximal portion 100 axially in a proximal direction or away from the handle assembly 44 (rightward relative to the orientation of FIG. 4). Proximal movement of the proximal portion 100 reduces compression in the inner shaft assembly 42; as a result, compression in the prosthetic heart valve 30 is also lessened. Throughout the delivery and deployment procedures, axial forces being experienced by the inner shaft assembly 40, and thus by the prosthetic heart valve 30, can be sensed and reviewed by the clinician, for example via the sensor unit 118 (referenced generally).

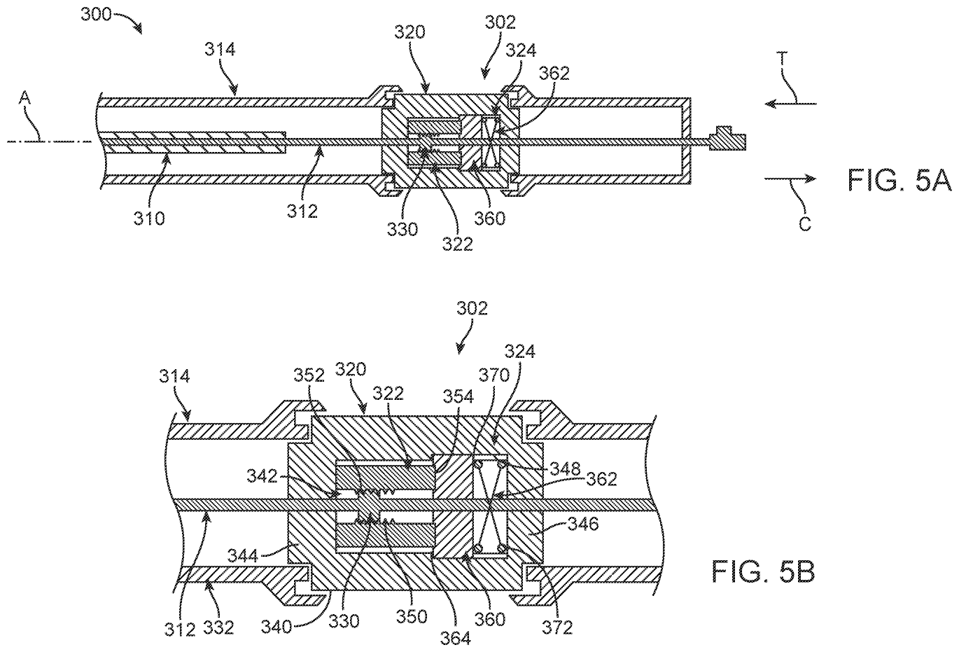

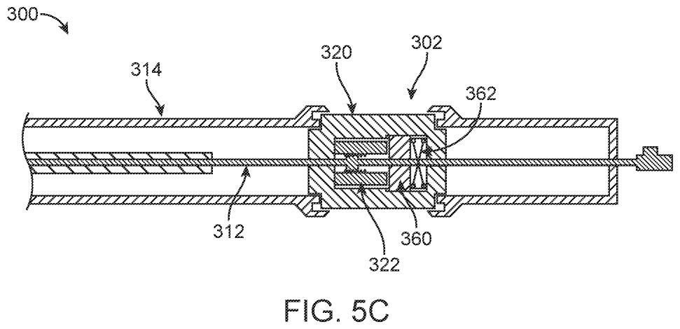

FIG. 5A illustrates, in simplified form, portions of a delivery device 300 including another embodiment axial force adjustment assembly 302 in accordance with principles of the present disclosure. As a point of reference, the delivery device 300 can generally assume any of the forms described elsewhere in the present disclosure, and additionally includes an outer shaft assembly 310 (e.g., akin to the outer shaft assembly 40 (FIG. 1)), an inner shaft assembly 312 (e.g., akin to the inner shaft assembly 42 (FIG. 1)), and a handle assembly 314 (e.g., akin to the handle assembly 44 (FIG. 1)). As with other embodiments, the axial force adjustment assembly 302 is operable to selectively alter an axial force (tension or compression) in or applied to the inner shaft assembly 312, for example by selectively moving a proximal section of the inner shaft assembly 312 relative to the handle assembly 314.

The axial force adjustment assembly 302 includes an actuator member 320, a driver member 322, and an optional interface sub-assembly 324. Details on the various components are provided below. In general terms, the actuator member 320 is rotatably connected to the handle assembly 314. The driver member 322 is connected to a proximal section 330 of the inner shaft assembly 312. The actuator member 320 is linked to the driver member 322, for example via the optional interface sub-assembly 324. With this construction, the proximal section 330 can be caused to translate or move along a center, longitudinal axis A defined by the inner shaft assembly 312 in response to a user-applied force at the actuator member 320. In some embodiments, the interface sub-assembly 324 serves as a clutch mechanism, limiting compression or tension that can be applied to the inner shaft assembly 312 by the axial force adjustment assembly 302.

With reference to FIG. 5B, the actuator member 320 can be a hub-like body, and includes or carries features appropriate for rotatable coupling to or with the handle assembly 314. For example, in the simplified representation of FIG. 5B, the handle assembly 314 includes a chassis or housing 332; the actuator member 320 can be coupled the chassis 332 in a manner permitting rotation of the actuator member 320 relative to the chassis 332 while maintaining an axial position of the actuator member 320 relative to the chassis 332 as would be apparent to one of ordinary skill. Regardless, the actuator member 320 defines a contact surface 340 that is exposed or accessible by a user upon final assembly. In some embodiments, the actuator member 320 forms or defines a cavity 342 between opposing, leading and trailing end walls 344, 346. As described in greater detail below, the actuator member 320 can further form or carry one more features at or along an inner surface 348 thereof for engaging a complementary feature(s) of the interface sub-assembly 324.

The driver member 322 is generally configured to interface with the proximal section 330 in a manner dictating an axial or longitudinal position of the proximal section 330, for example akin to a leadscrew design. In one non-limiting embodiment, the driver member 322 forms or defines an internally threaded surface 350 sized and shaped to threadably engage an externally threaded surface 352 of the proximal section 330. The driver member 322 is sized and shaped to be received within the cavity 342, and is captured within the cavity 342 as described below. In this regard, the driver member 322 can further form or carry one more features at or along a trailing end 354 thereof for engaging a complementary feature(s) of the interface sub-assembly 324.

The interface sub-assembly 324 can assume various forms, and in some embodiments includes an engagement member 360 and a biasing member 362. The engagement member 360 is sized to be received within the cavity 342, and includes one or more features that facilitate a rotationally captured arrangement relative to the actuator member 320. In particular, upon final assembly, connection between the actuator member 320 and the engagement member 360 is such that engagement member 360 rotates with rotation of the actuator member 320, but can slide axially or longitudinally relative to the actuator member 320. For example, the inner surface 348 of the actuator member 320 and an outer surface of the engagement member 360 can form complementary, axial or longitudinal slots and ribs that achieve rotational engagement while permitting the engagement member 360 to translate axially.

The engagement member 360 further includes one or more features that facilitate selective engagement with the driver member 322 such that when engaged, a rotational force on the engagement member 360 is transferred to the driver member 322 (and vice-versa). For example, a leading side 364 of the engagement member 360 can form a toothed surface complementary with a toothed surface formed at the trailing end 354 of the driver member 322. When the engagement member 360 is forced axially toward the driver member 322, the toothed surfaces mesh with one another such that a rotational force of the engagement member 360 is transferred to the driver member 322. As the axial force on the engagement member 360 toward the driver member 322 is reduced, the toothed surfaces no longer mesh and the driver member 322 does not rotate with rotation of the engagement member 360.

The biasing member 362 can assume various forms and in some embodiments is, or is akin to, a coil or compression spring. The biasing member 362 is sized to be received within the cavity 342, and defines a first end 370 opposite a second end 372. A spring force constant of the biasing member 362 is selected to dictate an upper limit on a forward compression force that can be generated by the axial force adjustment assembly 302 as described in greater detail below.

Construction of the axial force adjustment assembly 302 includes the actuator member 320 rotatably assembled to the handle assembly chassis 332. The biasing member 362 is disposed within the cavity 342, with the second end 372 bearing against the trailing end wall 346. The engagement member 360 is coupled to the actuator member 320 within the cavity 342 as described above (i.e., such that the engagement member 360 can slide axially relative to the actuator member 320, but rotates with rotation of the actuator member 320), immediately adjacent the biasing member 362 (i.e., a side of the engagement member 360 faces and is in contact with the first end 370 of the biasing member 362). The driver member 322 is threadably coupled to the proximal section 330 of the inner shaft assembly 312. The driven member 322 is arranged in the cavity 342 adjacent the leading end wall 344, with the trailing end 354 facing the leading side 364 of the engagement member 360. As reflected by FIG. 5B, various components of the axial force adjustment assembly 302 can form or define requisite openings or passages to receive and allow axial movement of the inner shaft assembly 312.

With reference to FIGS. 5A and 5B, upon final assembly, the axial force adjustment assembly 302 axially or longitudinally retains the proximal portion 330 of the inner shaft assembly 312 relative to the handle assembly 314 (and thus relative to the outer shaft assembly 310 that is otherwise secured to the handle assembly 314) via axial or longitudinal connections between the proximal portion 330/driver member 322, the driver member 322/engagement member 360, the engagement member 360/biasing member 362/a ctuator member 320, and the actuator member 320/handle assembly 314. When the biasing member 362 forces the engagement member 360 into meshed engagement with the driver member 322, rotation of the actuator member 320 is transferred to the driver member 322; rotation of the driver member 322, in turn, translates into axial movement of the inner shaft assembly 312. When compression in the inner shaft assembly 312 exceeds a spring force constant or stiffness of the biasing member 362, robust meshed engagement between the driver member 322 and the engagement member 360 no longer exists; under these circumstances, while the engagement member 360 will rotate with rotation of the actuator member 320, the toothed surface of the engagement member 360 will slide over the toothed surface of the driver member 322 thereby limiting compression forces that can be applied by the axial force adjustment assembly 302 onto the inner shaft assembly 312.

The axial force adjustment assembly 302 can be operated to lessen the tension or compression in the inner shaft assembly 312 by axially or longitudinally moving the proximal portion 330 relative to the handle assembly 314. For example, compression in the inner shaft assembly 312 imparts a compression force C at the proximal portion 330/driver member 322 interface, and can be lessened by rotating the actuator member 320 in a direction that causes the driver member 322 to move the proximal portion 330 axially in a proximal direction relative to the handle assembly 314 (rightward relative to the orientation of FIG. 5A). Conversely, tension in the inner shaft assembly 312 imparts a tension force T at the proximal portion 330/driver member 322 interface, and can be lessened by rotating the actuator member 320 in a direction that causes the driver member 322 to move the proximal portion 330 axially in a distal direction relative to the handle assembly 314 (leftward relative to the orientation of FIG. 5A). In this regard, rotation of the driver member 322 in response to rotation of the actuator member 320 will continue until the force required to drive the proximal portion 330 in the distal direction exceeds the spring force constant or stiffness of the biasing member 362 as in FIG. 5C. Under these circumstances, the toothed surface 364 (FIG. 5B) of the engagement member 360 will slide over the toothed surface 354 (FIG. 5B) of the driven member 322, effectively limiting compression that can be applied by the axial force adjustment assembly 302 onto the inner shaft assembly 312 as part of the tension-reduction operation. Regardless, because axial forces (tension/compression) in the inner shaft assembly 312 can be directly related to or generated as a function of tension/compression at the prosthetic heart valve 30 (FIG. 2) during a delivery procedure, adjustment or lessening of axial forces on the inner shaft assembly 312 via operation of the axial force adjustment assembly 302 equates to a reduction of tension/compression in the prosthetic heart valve 30 as described above.

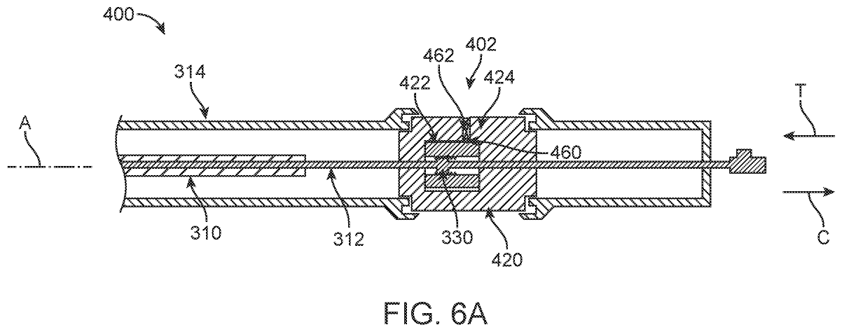

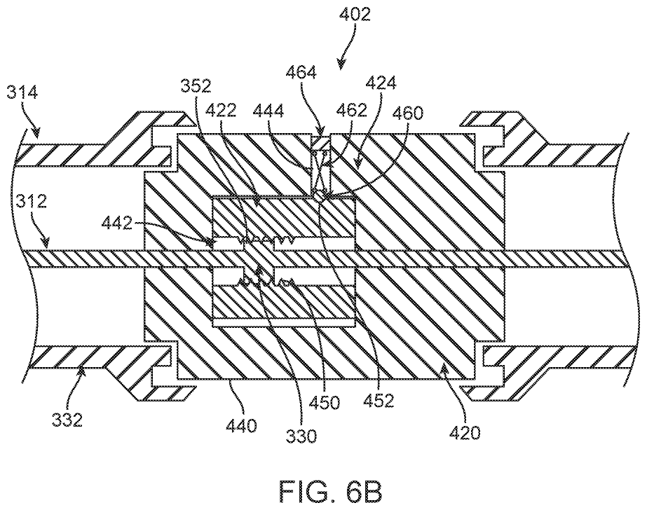

FIG. 6A illustrates, in simplified form, portions of a delivery device 400 including another embodiment axial force adjustment assembly 402 in accordance with principles of the present disclosure. As a point of reference, the delivery device 400 can be similar to the delivery device 300 (FIG. 5A), and additionally includes the outer shaft assembly 310, the inner shaft assembly 312, and the handle assembly 314. As with other embodiments, the axial force adjustment assembly 402 is operable to selectively alter an axial force (tension or compression) in or applied to the inner shaft assembly 312, for example by selectively moving a proximal section of the inner shaft assembly 312 relative to the handle assembly 314.

The axial force adjustment assembly 402 includes an actuator member 420, a driver member 422, and an optional interface sub-assembly 424 (referenced generally). Details on the various components are provided below. In general terms, the actuator member 420 is rotatably connected to the handle assembly 314. The driver member 422 is connected to the proximal section 330 of the inner shaft assembly 312. The actuator member 420 is linked to the driver member 422, for example via the optional interface sub-assembly 424. With this construction, the proximal section 330 can be caused to translate or move along the center, longitudinal axis A defined by the inner shaft assembly 312 in response to a user-applied force at the actuator member 420. In some embodiments, the interface sub-assembly 424 serves as a clutch mechanism, limiting compression or tension that can be applied to the inner shaft assembly 312 by the axial force adjustment assembly 402.

With reference to FIG. 6B, the actuator member 420 can be a hub-like body, and includes or carries features appropriate for rotatable coupling to or with the handle assembly 314 (e.g., the actuator member 420 can be coupled the chassis 332 in a manner permitting rotation of the actuator member 420 relative to the chassis 332 while maintaining an axial position of the actuator member 420 relative to the chassis 332). Regardless, the actuator member 420 defines a contact surface 440 that is exposed or accessible by a user upon final assembly. In some embodiments, the actuator member 420 forms or defines a cavity 442, and one or more radial bores 444 open to the cavity 442 for reasons made clear below.

The driver member 422 can be akin to the driver member 322, generally configured to interface with the proximal section 330 in a manner dictating an axial or longitudinal position of the proximal section 330, for example akin to a leadscrew design. In one non-limiting embodiment, the driver member 422 forms or defines an internally threaded surface 450 sized and shaped to threadably engage the externally threaded surface 352 of the proximal section 330. The driver member 422 is sized and shaped to be received within the cavity 442, and is captured within the cavity 442 as described below. Further, the driver member 422 forms or defines one or more detents 452 for reasons made clear below.

The interface sub-assembly 424 can assume various forms, and in some embodiments includes an engagement member 460 and a biasing member 462. The engagement member 460 can be a ball or sphere, and is sized to be selectively received within the detent 452 (e.g., a diameter of the detent 452 is slightly smaller than the ball 460). The biasing member 462 can be a compression spring or the like, sized to be received within the radial bore 444 so as to bear against the engagement member 460. The biasing member 462 can be retained within the radial bore 444 in various manners, and in some embodiments a fitting 464 is provided. The biasing member 462 thus bears against the fitting 464 and the engagement member 460, applying a spring force onto the engagement member 460. In some embodiments, a position of the fitting 464 relative to the radial bore 444 is adjustable (e.g., the fitting 464 is a screw component that is threaded to the actuator member 420), thereby facilitating manual adjustment or setting of the biasing member 462 to a desired force. While a single engagement member 460/biasing member 462 pair is shown in FIGS. 6A and 6B, additional pairs can be provided (with a corresponding number of radial bores 444 and detents 452).

Upon final assembly, the driver member 422 is rotatably captured within the cavity 442 (e.g., the actuator member 420 can rotate relative to the driver member 422 and vice-versa, but axial movement of the driver member 422 is limited by walls of the actuator member 420), with the detent 452 being axially aligned with the radial bore 444. As the actuator member 420 is rotated relative to the driver member 422, the radial bore 444 (and thus the biasing member 462 carried by the radial bore 444 and the engagement member 460 carried by the biasing member 462), is brought into radial alignment with the detent 452 (i.e., the actuator member 420 can be rotationally positioned such that the radial bore 444 is open to the detent 452). The biasing member 462 biases the engagement member 460 toward driver member 422. When the radial bore 444 is aligned with the detent 452, the engagement member 460 is biased into the detent 452, thus linking or connecting the actuator member 420 with the driver member 422. When so-connected, the driver member 422 rotates with rotation of the actuator member 420.

With reference between FIGS. 6A and 6B, during use, the axial force adjustment assembly 402 axially or longitudinally retains the proximal portion 330 of the inner shaft assembly 312 relative to the handle assembly 314 (and thus relative to the outer shaft assembly 310 that is otherwise secured to the handle assembly 314) via axial or longitudinal connections between the proximal portion 330/driver member 422, the driver member 422/actuator member 420, and the actuator member 420/handle assembly 314. When the biasing member 462 forces the engagement member 460 into engagement with the detent 452 of the driver member 422, rotation of the actuator member 420 is transferred to the driver member 422; rotation of the driver member 422, in turn, translates into axial movement of the inner shaft assembly 312. When the force required to drive the inner shaft assembly 312 exceeds a spring force constant or stiffness of the biasing member 462, the engagement member 460 disengages from the detent 452 with further rotation of the actuator member 420; under these circumstances, further rotation of the actuator member 420 will no longer drive the inner shaft assembly 312 as the driver member 422 does not rotate with the actuator member 420 thereby limiting torque forces that can be applied by the axial force adjustment assembly 402 and in turn translated to axial forces onto the inner shaft assembly 312.

The axial force adjustment assembly 402 can be operated to lessen the tension or compression in the inner shaft assembly 312 by axially or longitudinally moving the proximal portion 330 relative to the handle assembly 314. For example, compression in the inner shaft assembly 312 imparts a compression force C at the proximal portion 330/driver member 422 interface, and can be lessened by rotating the actuator member 420 in a direction that causes the driver member 422 to move the proximal portion 330 axially in a proximal direction relative to the handle assembly 314 (rightward relative to the orientation of FIG. 6A). Conversely, tension in the inner shaft assembly 312 imparts a tension force T at the proximal portion 330/driver member 422 interface, and can be lessened by rotating the actuator member 420 in a direction that causes the driver member 422 to move the proximal portion 330 axially in a distal direction relative to the handle assembly 314 (leftward relative to the orientation of FIG. 6A). In this regard, rotation of the driver member 422 in response to rotation of the actuator member 420 will continue until the applied forward compression force exceeds the spring force constant or stiffness of the biasing member 462. Under these circumstances, the engagement member 460 will disengage from the driver member 422, effectively limiting compression or tension that can be applied by the axial force adjustment assembly 402 onto the inner shaft assembly 312 as part of tension-reduction operation. Regardless, because axial forces (tension/compression) in the inner shaft assembly 312 can be directly related to or generated as a function of tension/compression at the prosthetic heart valve 30 (FIG. 2) during a delivery procedure, adjustment or lessening of axial forces on the inner shaft assembly 312 via operation of the axial force adjustment assembly 402 equates to a reduction of tension/compression in the prosthetic heart valve 30 as described above

Returning to FIGS. 1 and 2, some aspects of the present disclosure relate to a delivery devices configured to sense axial forces (tension or compression) along the inner shaft assembly 42, and display information indicative of the sensed axial forces to a user. For example, some delivery devices of the present disclosure can include the sensor unit 118 with or without an axial force adjustment assembly. Other sensor constructions are also envisioned for enabling a clinician to understand tension/compression in the system, and that can be provided with or without the axial force adjustment assemblies of the present disclosure. For example, a tension/compression sensor can be incorporated into the delivery device to measure the forces in the inner shaft assembly 42 (i.e., the shaft assembly to which the stented prosthetic heart valve 30 is secured). The sensor may be located along a distal region of the delivery device, for example proximate the valve retainer 72 (e.g., within 5 centimeters of the valve retainer 72) and engineered with an electronic circuit to provide an output reading at the handle assembly 44. Alternatively, a mechanical gauge can be connected to the proximal end of the delivery device (e.g., akin to the sensor unit 118).

The delivery devices of the present disclosure can be used with a variety of stented prosthetic heart valve constructions. In general terms, the stented prosthetic heart valves of the present disclosure include a stent or stent frame having an internal lumen maintaining a valve structure (tissue or synthetic), with the stent frame having a normal, expanded condition or arrangement and collapsible to a compressed condition or arrangement for loading within a delivery device. The stent frame is normally constructed to self-deploy or self-expand when released from the delivery device. For example, the stents or stent frames are support structures that comprise a number of struts or wire segments arranged relative to each other to provide a desired compressibility and strength to the prosthetic heart valve. The struts or wire segments are arranged such that they are capable of self-transitioning from a compressed or collapsed condition to a normal, radially expanded condition. The struts or wire segments can be formed from a shape memory material, such as a nickel titanium alloy (e.g., nitinol). The stent frame can be laser-cut from a single piece of material, or can be assembled from a number of discrete components.

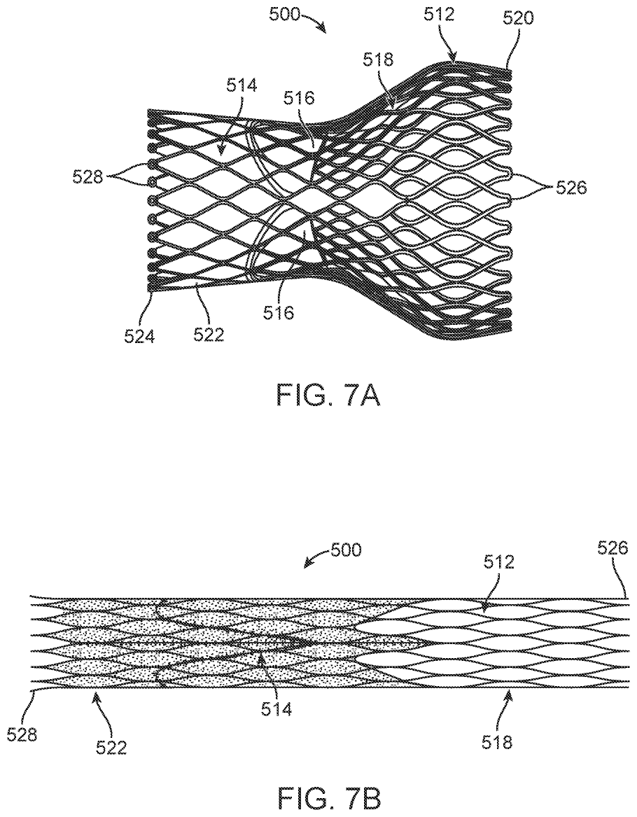

With the above understanding in mind, one simplified, non-limiting example of a stented prosthetic heart valve 500 useful with systems, devices and methods of the present disclosure is illustrated in FIG. 7A. As a point of reference, the prosthetic heart valve 500 is shown in a normal or expanded condition in the view of FIG. 7A; FIG. 7B illustrates the prosthetic heart valve in a compressed condition (e.g., when compressively retained within an outer catheter or sheath as described below). The prosthetic heart valve 500 includes a stent or stent frame 512 and a valve structure 514. The stent frame 512 can assume any of the forms mentioned above, and is generally constructed so as to be self-expandable or balloon-expandable from the compressed condition (FIG. 7B) to the normal, expanded condition (FIG. 7A). In some embodiments, the stent frame can be balloon expandable or expanded mechanically.

The valve structure 514 can assume a variety of forms, and can be formed, for example, from one or more biocompatible synthetic materials, synthetic polymers, autograft tissue, homograft tissue, xenograft tissue, or one or more other suitable materials. In some embodiments, the valve structure 514 can be formed, for example, from bovine, porcine, equine, ovine and/or other suitable animal tissues. In some embodiments, the valve structure 514 can be formed, for example, from heart valve tissue, pericardium, and/or other suitable tissue. In some embodiments, the valve structure 514 can include or form one or more leaflets 516. For example, the valve structure 514 can be in the form of a tri-leaflet valve, a bi-leaflet valve, or another suitable valve. In some constructions, the valve structure 514 can comprise two or three leaflets that are fastened together at enlarged lateral end regions to form commissural joints, with the unattached edges forming coaptation edges of the valve structure 514. The leaflets 516 can be fastened to a skirt that in turn is attached to the frame 512. The upper ends of the commissure points can define an inflow portion 518 corresponding to a first or inflow end 520 of the prosthetic heart valve 500. The opposite end of the valve can define an outflow portion 522 corresponding to a second or outflow end 524 of the prosthetic heart valve 510. As shown, the stent frame 512 can have a lattice or cell-like structure, and optionally forms or provides crowns 526 and/or eyelets 528 (or other shapes) at the outflow and inflow ends 520, 524.

With the one exemplary construction of FIGS. 7A and 7B, the prosthetic heart valve 500 can be configured (e.g., sized and shaped) for replacing or repairing an aortic valve. Alternatively, other shapes are also envisioned, adapted to mimic the specific anatomy of the valve to be repaired (e.g., stented prosthetic heart valves useful with the present disclosure can alternatively be shaped and/or sized for replacing a native mitral, pulmonic or tricuspid valve or compassionate use such as heterotopic implants).

Although the present disclosure has been described with reference to preferred embodiments, workers skilled in the art will recognize that changes can be made in form and detail without departing from the spirit and scope of the present disclosure.

Description claims:

1. A delivery device for percutaneously delivering a stented prosthetic heart valve, the delivery device comprising:
an inner shaft assembly including a valve retainer configured for temporary connection to a stented prosthetic heart valve in a loaded state of the delivery device;
an outer shaft assembly co-axially received over the inner shaft assembly and including a capsule configured to contain the stented prosthetic heart valve in the loaded state;
a handle assembly coupled to a proximal region of the outer shaft assembly; and
an axial force adjustment assembly connecting a proximal section of the inner shaft assembly to the handle assembly, the axial force adjustment assembly configured to selectively move the proximal section relative to the handle assembly.

2. The delivery device of claim 1, wherein the inner shaft assembly defines a longitudinal axis, and further wherein the axial force adjustment assembly is configured to selectively move the proximal section axially along the longitudinal axis.

3. The delivery device of claim 1, wherein the axial force adjustment assembly is configured to selectively alter an axial force in the inner shaft assembly, the axial force being one of tension and compression.

4. The delivery device of claim 1, wherein the axial force adjustment assembly includes an actuator member linked to a driver member, and further wherein the driver member directly interfaces with the proximal section of the inner shaft assembly, and ever further wherein the axial force adjustment assembly is configured to selectively transfer a force applied to the actuator member onto the proximal section via the driver member.

5. The delivery device of claim 4, wherein the axial force adjustment assembly is further configured to not transfer a force applied to the actuator member onto the proximal section via the driver member when a longitudinal force in the inner shaft assembly exceeds a predetermined level.

6. The delivery device of claim 4, wherein the actuator member is rotatably connected to the handle assembly, and further wherein the axial force adjustment assembly is configured to translate a rotational force applied to the actuator member into longitudinal movement of the proximal section.

7. The delivery device of claim 6, wherein the inner shaft assembly includes a shaft maintaining a threaded surface, and further wherein the driver member defines a complementary threaded surface, the complementary threaded surface in meshed engagement with the threaded surface.

8. The delivery device of claim 7, wherein the actuator member is selectively coupled to the driver member by an interface sub-assembly including an engagement member and a biasing member.

9. The delivery device of claim 8, wherein the engagement member includes a toothed surface, and further wherein the biasing member biases the toothed surface into meshed engagement with a complementary toothed surface of the driver member when a force applied to the engagement member is less than a spring force constant of the biasing member.

10. The delivery device of claim 8, wherein the engagement member includes a ball, and further wherein the biasing member biased the ball into captured engagement with a complementary aperture in the driver member when a force applied to the engagement member is less than a spring force constant of the biasing member.

11. The delivery device of claim 1, further comprising a sensor configured and arranged to sense a parameter indicative of an axial force on the inner shaft assembly.

12. The delivery device of claim 11, wherein the sensor is located proximate the valve retainer.

13. The delivery device of claim 11, further comprising an output device carried by the handle assembly and configured to display an axial force reading based upon information signaled by the sensor.

14. A method for restoring a defective heart valve in a patient, the method comprising:
manipulating a delivery device loaded with a radially expandable stented prosthetic heart valve in a radially compressed condition, the delivery device including an outer shaft assembly including a capsule containing the stented prosthetic heart valve, an inner shaft assembly co-axially disposed within the outer shaft assembly and including a valve retainer connected to a proximal segment of the stented prosthetic heart valve, a handle assembly coupled to a proximal region of the outer shaft assembly, and an axial force adjustment assembly connecting a proximal section of the inner shaft assembly to the handle assembly;
wherein the step of manipulating includes guiding the stented prosthetic heart valve through a vasculature of the patient and into the defective heart valve by moving the handle assembly, which correspondingly moves the outer shaft assembly and the inner shaft assembly;
partially retracting the capsule to expose a distal segment of the stented prosthetic heart valve such that the exposed distal segment radially expands;
wherein the steps of manipulating and retracting generates an axial force in the inner shaft assembly;
operating the axial force adjustment assembly to lessen the axial force in the inner shaft assembly; and
releasing the stented prosthetic heart valve from the delivery device.

15. The method of claim 14, wherein the step of operating include rotating an actuator member of the axial force adjustment assembly.

16. The method of claim 14, wherein the step of operating includes moving the proximal section axially relative to the handle assembly.

17. The method of claim 14, wherein following the step of partially retracting, the exposed distal segment engages native anatomy at the defective heart valve and generates stress in the stented prosthetic heart valve, and further wherein a component force of the stress is transferred to the inner shaft assembly via the valve retainer to generate the axial force in the inner shaft assembly.

18. The method of claim 14, wherein prior to the step of operating, the method further comprising:
evaluating an axial force in the inner shaft assembly based upon information from a sensor located along the inner shaft assembly.

19. A delivery device for percutaneously delivering a stented prosthetic heart valve, the delivery device comprising:
an inner shaft assembly including a valve retainer configured for temporary connection to a stented prosthetic heart valve in a loaded state of the delivery device;
an outer shaft assembly co-axially received over the inner shaft assembly and including a capsule configured to contain the stented prosthetic heart valve in the loaded state;
a handle assembly coupled to a proximal region of the outer shaft assembly; and
a sensor configured and arranged to sense a parameter indicative of an axial force in the inner shaft assembly.

20. The delivery device of claim 19, wherein the sensor is located proximate the valve retainer.
